(19) United States
(12) Patent Application Publication
(10) Pub. No.: US 2025/0166742 A1
(43) Pub. Date: May 22, 2025

(54) VIRTUAL MATERIAL DESIGN SERVICE FRAMEWORK FOR DEVELOPING MATERIAL FOR SECONDARY BATTERY BASED ON MICROSERVICE ARCHITECTURE AND OPERATING METHOD THEREOF

(71) Applicant: Korea Electronics Technology Institute, Seongnam-si (KR)

(72) Inventors: Sang Yub LEE, Yongin-si (KR); Inpyo CHO, Yongin-si (KR); Jaekyu LEE, Yongin-si (KR)

(21) Appl. No.: 18/656,960

(22) Filed: May 7, 2024

(30) Foreign Application Priority Data
Nov. 16, 2023 (KR) 10-2023-0159609

(51) Int. Cl. G16C 60/00; G06V 10/82; G16C 20/90

(52) U.S. Cl. CPC G16C 60/00; G06V 10/82; G16C 20/90

(57) ABSTRACT

Proposed is a virtual material design service framework system for developing a material for a secondary battery based on a microservice architecture. The system may include a user interface unit receiving information on a new material having at least one structure of a molecular structure, a microstructure, and a cell structure, an experiment database unit storing experiment data information corresponding to the information on the new material, and a simulation unit generating a simulation model by performing simulations on the received information on the new material. The system may also include a data modeling unit generating a data model based on the experiment data information, a combination modeling unit combining the simulation model and the data model according to a combination modeling scheme, and an artificial intelligence service unit converting the combination model into an image and deriving a candidate material based on a predetermined image recognition scheme.

### CROSS-REFERENCE TO RELATED APPLICATION

This application claims priority to and the benefit of Korean Patent Application No. 10-2023-0159609 filed on Nov. 16, 2023, the disclosure of which is incorporated herein by reference in its entirety.

### BACKGROUND

The present disclosure relates to a virtual material design service framework for developing a material for a secondary battery based on a microservice architecture and an operating method thereof.

### SUMMARY

One aspect is a virtual material design service framework for developing a material for a secondary battery based on a microservice architecture, which can provide a microservice architecture in which results that are performed in stages can be checked simultaneously when a new material for a secondary battery is developed and can minimize time and costs for the entire process through an artificial intelligence model capable of integrating and using data through the microservice architecture, and an operating method of the virtual material design service framework.

However, aspects of the present disclosure to be achieved are not limited to the aspects disclosed herein, and other aspects may be present.

Another aspect is a virtual material design service framework system for developing a material for a secondary battery based on a microservice architecture that includes a user interface unit configured to receive information on a new material having at least one structure of a molecular structure, a microstructure, and a cell structure, an experiment database unit in which experiment data information corresponding to the information on the new material has been stored, a simulation unit configured to generate a simulation model by performing simulations on the received information on the new material, a data modeling unit configured to generate a data model based on the experiment data information, a combination modeling unit configured to combine the simulation model and the data model according to a combination modeling scheme, and an artificial intelligence service unit configured to convert the combination model into an image and to derive a candidate material based on a predetermined image recognition scheme.

Another aspect is an operating method of a virtual material design service framework for developing a material for a secondary battery based on a microservice architecture that includes receiving information on a new material having at least one structure of a molecular structure, a microstructure, and a cell structure, generating a simulation model by performing simulations on the received information on the new material, generating a data model based on experiment data information corresponding to the information on the new material, generating a combination model by combining the simulation model and the data model according to a combination modeling scheme, and converting the combination model into an image and deriving a candidate material through a pre-trained artificial intelligence model to which a predetermined image recognition scheme has been applied.

In addition, another method and another system for implementing an embodiment of the present disclosure, and a computer-readable recording medium on which a computer program for executing the method has been recorded may be further provided.

According to the embodiment of the present disclosure, data at various scales of a molecular structure, a microstructure, and a cell (or system) unit can be secured, integrated, and analyzed in order to secure a new material. This enables comprehensive information necessary to discover and develop a new material to be obtained and also enables an integrated analysis service.

Furthermore, an artificial intelligence integrated engine for each stage is provided by using the microservice architecture, which provides an environment in which the artificial intelligence integrated engine is easy to be continuously improved. In particular, it is possible to provide higher accuracy to the research and development of a material by providing a system function for improving accuracy by using an image-based artificial intelligence model.

Furthermore, it is possible to provide a method of collecting data in a condition in which it is difficult to collect actual data through combination modeling. The method presents an alternative for obtaining a dataset necessary for the artificial intelligence model and enabling limits in the research of a material to be overcome.

In addition, it is possible to provide a service capable of reducing costs and time compared to the existing method by applying an integrated analysis function and an advanced artificial intelligence model.

Effects of the present disclosure which may be obtained in the present disclosure are not limited to the aforementioned effects, and other effects not described above may be evidently understood by a person having ordinary knowledge in the art to which the present disclosure pertains from the following description.

### BRIEF DESCRIPTION OF THE DRAWINGS

FIG. 1 is a diagram for describing the conventional contents of simulations for each scale unit.

FIG. 2 is a diagram illustrating a comparison with a common software structure.

FIG. 3 is a diagram for describing the concept of a combination modeling scheme.

FIGS. 4A to 4C are diagrams illustrating an architecture structure for each unit according to a conventional technology.

FIG. 5 is a functional block diagram of a virtual material design service framework system according to an embodiment of the present disclosure.

FIGS. 6 and 7 are diagrams for describing an aspect in which data are used in a conventional technology and an embodiment of the present disclosure.

FIG. 8 is a flowchart of an operating method of a virtual material design service framework according to an embodiment of the present disclosure.

FIG. 9 is a block diagram of a construction of the virtual material design service framework system according to an embodiment of the present disclosure.

### DETAILED DESCRIPTION

The following problems are present in discovering a new material for improving performance of a secondary battery and solving a problem with the secondary battery.

First, in order to discover a new material, various material combinations need to be generated and the stability thereof needs to be determined in a molecular structure stage. To this end, the new material needs to be estimated by using a computational chemistry method. However, the computational chemistry method consumes a lot of time and costs because results need to be derived through meta-heuristic calculation and repeated experiments.

Second, separate experiments need to be performed in a microstructure stage in order to evaluate the suitability of a new material although the new material is selected by using an artificial intelligence scheme (e.g., a classification model or an inference model) in the molecular structure stage.

Third, in the microstructure stage, a task for checking whether a developed material satisfies adsorption, transmission, and porosity characteristics is performed. Such an experimental task is complicated, and may require an additional task for evaluating, improving, and optimizing the characteristics of a material through experiments.

Finally, a cell (or system) structure stage has a problem in that the evaluation of electrical and chemical characteristics needs to be separately performed so that a cell characteristic of a material that satisfies both the molecular structure stage and the microstructure stage can be determined.

Advantages and characteristics of the present disclosure and a method for achieving the advantages and characteristics will become apparent from the embodiments described in detail later in conjunction with the accompanying drawings. However, the present disclosure is not limited to embodiments disclosed hereinafter, but may be implemented in various different forms. The embodiments are merely provided to complete the present disclosure and to fully notify a person having ordinary knowledge in the art to which the present disclosure pertains of the category of the present disclosure. The present disclosure is merely defined by the claims.

Terms used in this specification are used to describe embodiments and are not intended to limit the present disclosure. In this specification, an expression of the singular number includes an expression of the plural number unless clearly defined otherwise in the context. The term “comprises” and/or “comprising” used in this specification does not exclude the presence or addition of one or more other elements in addition to a mentioned element. Throughout the specification, the same reference numerals denote the same elements. “And/or” includes each of mentioned elements and all combinations of one or more of mentioned elements. Although the terms “first”, “second”, etc. are used to describe various components, these elements are not limited by these terms. These terms are merely used to distinguish between one element and another element. Accordingly, a first element mentioned hereinafter may be a second element within the technical spirit of the present disclosure.

All terms (including technical and scientific terms) used in this specification, unless defined otherwise, will be used as meanings which may be understood in common by a person having ordinary knowledge in the art to which the present disclosure pertains. Furthermore, terms defined in commonly used dictionaries are not construed as being ideal or excessively formal unless specially defined otherwise.

Hereinafter, in order to help understanding of those skilled in the art, a proposed background of the present disclosure is described in detail.

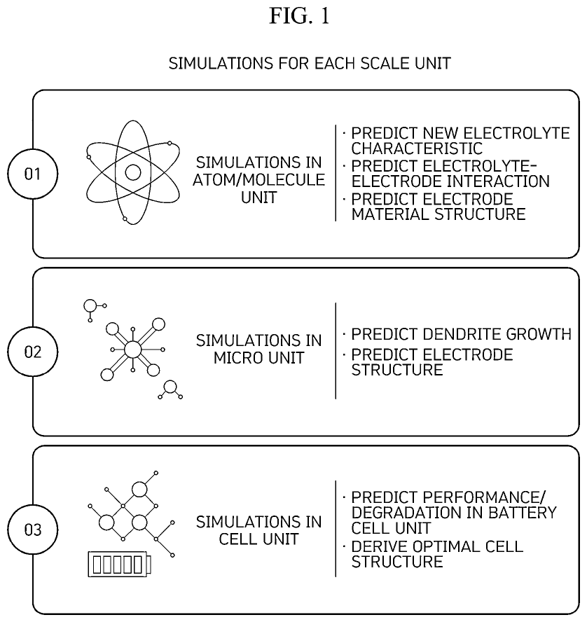

FIG. 1 is a diagram for describing the conventional contents of simulations for each scale unit. (Source: Virtual Material Design Technology, the innovation of the development of a new material that is performed in a virtual space, the development of a battery in Hyundai Motor's Basic Research Institute)

Conventionally, as illustrated in FIG. 1, data in each unit are secured by dividing the data into a molecular structure unit, a microstructure unit, and a cell (or system) structure unit. A new material is discovered by applying an artificial intelligence (AI) scheme to the data. Such a method may not satisfy the suitability of data in a next stage although the suitability is satisfied in the molecular structure unit. In this case, the method has a fatal problem because a new material has to be reselected and calculated from the beginning again.

In order to solve such a problem, an embodiment of the present disclosure has an object of providing a service framework capable of connecting, integrating, and analyzing data for each unit by using a microservice architecture.

In this case, the microservice architecture refers to a software structure for constructing one application by combining a plurality of software components (or services) that are independently developed and executed. In order to divide an application into independent software services by using the microservice architecture and to process one request, the software services may constitute a distributed computing environment in which communication is performed through REST or messaging.

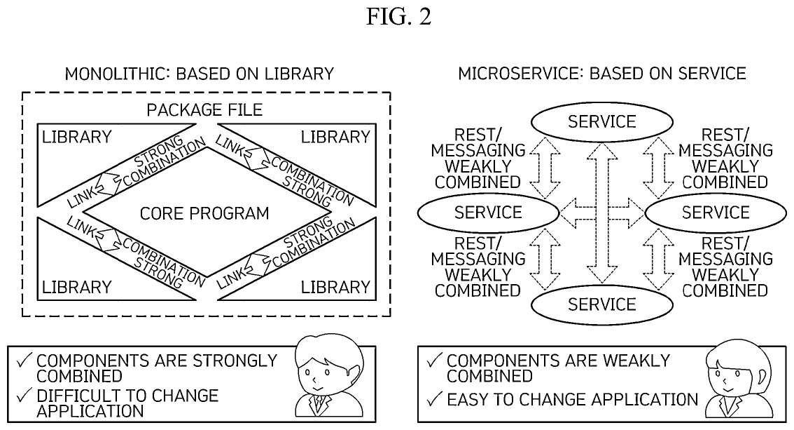

FIG. 2 is a diagram illustrating a comparison with a common software structure. (Source: Microservice Structure of Studying With Pictures)

The microservice architecture makes it easy to enhance flexibility and scalability compared to a monolithic architecture. In the microservice architecture, a tool and a language that are most suitable for each service can be selected by using various technical stacks. The microservice architecture is one of methods that are useful in efficiently managing and developing a modern large application and a complicated system.

An embodiment of the present disclosure is constructed so that a virtual material design scheme can be used based on the microservice architecture. In this case, the virtual material design scheme is a technology for simulating and predicting a unique characteristic and interaction in the molecular unit of a material and helps understanding of physical, chemical, and electrical characteristics of the material. Accordingly, the physical property of the material can be predicted, the stability of the material can be analyzed, and interaction modeling for the material is possible. Furthermore, in the virtual material design, the characteristics of a material can be predicted and optimized by using a large amount of experiment data and theoretical modeling through a combination of big data and an AI technology.

In this case, an embodiment of the present disclosure provides a service framework for the virtual material design by using the microservice architecture. In particular, an embodiment of the present disclosure provides a combination modeling function specified for image-based data processing. Accordingly, a microscopic characteristic of a material can be easily checked and analyzed through an image.

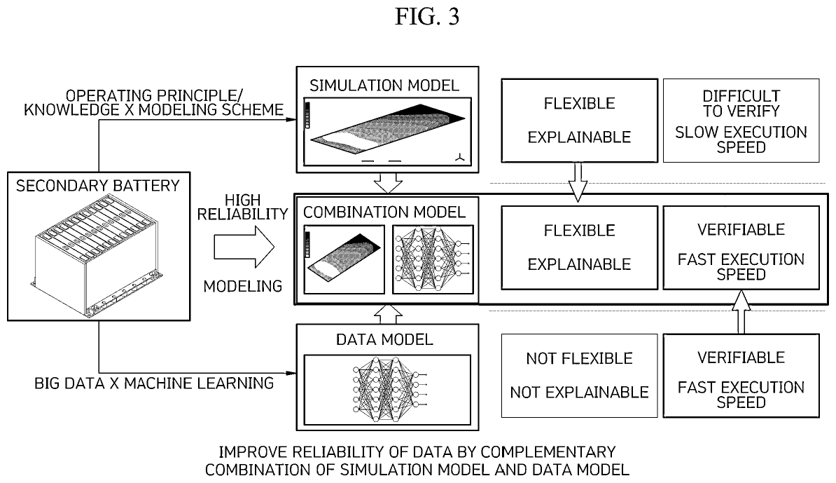

FIG. 3 is a diagram for describing the concept of the combination modeling scheme.

Conventionally, an AI model is generated and inferred by using a calculation method adapted to a computational chemical formula by using formulaic experiment data (or numbers) and a graphic neural network (GNN) algorithm using the calculation method. Such a conventional method has problems in that the amount of data is small and corresponding contents need to be verified through separate experiments in order to verify data matching.

In order to solve such problems, in an embodiment of the present disclosure, the combination modeling scheme capable of expanding the amount of data by converting the data into metadata associated with an image by using the existing experiment data and the results of computational analysis and improving the accuracy of a model is applied.

In this case, the combination modeling scheme is a method of improving the reliability of data by combining actual data and a simulation model based on knowledge, and is a method of improving the reliability of data through a mutual complementary combination of the simulation model and a data model.

As described above, an embodiment of the present disclosure constructs a service framework using the microservice architecture for integration based on the virtual material design scheme for each unit, but provides the entire system using the combination modeling scheme within the microservice architecture in order to discover a more accurate material.

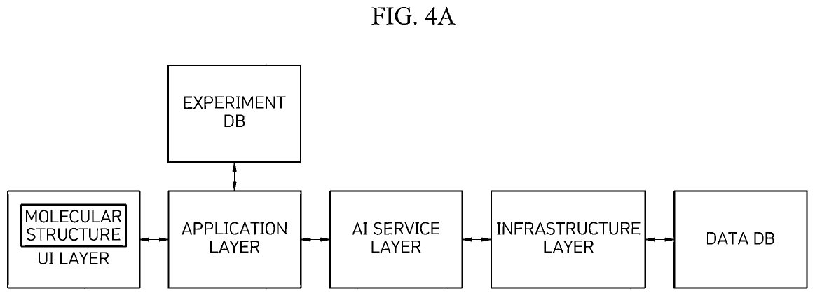

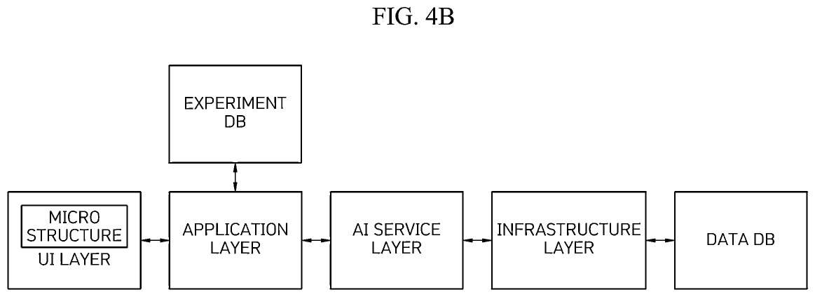

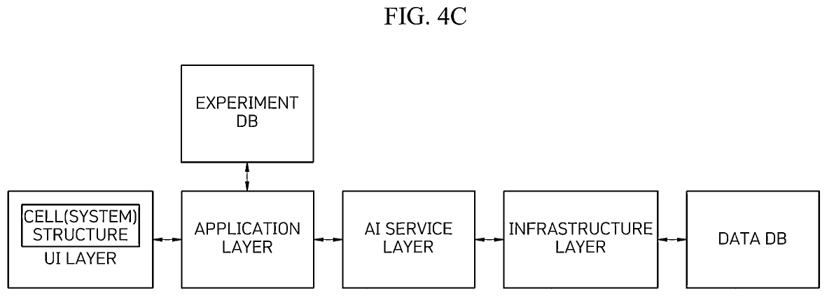

FIGS. 4A to 4C are diagrams illustrating an architecture structure for each unit according to a conventional technology.

As illustrated in FIGS. 4A to 4C, the architecture structure for each unit experiences a process of performing an input from a user through an application layer and collecting required experiment data. The architecture structure is constructed in a form in which an AI service model is developed based on the collected data, the results of the development are transmitted to a cloud service through an infrastructure layer, and corresponding results are stored.

In such a conventional technology, the unit structures independently operate through respective application layers, and do not share data. Accordingly, each unit independently collects data, trains a model, and does not share data and knowledge with another unit. This leads to the redundant collection of data and the absence of distributed knowledge.

Furthermore, if each unit structure independently develops a model and derives results, there is a problem in that it is difficult to guarantee whether such results are actually mutually engaged and suitable. If the coordination of data and models between the unit structures is not guaranteed, it is very difficult to obtain consistent results in the entire system.

Furthermore, if combination modeling or an advanced image-based AI model is independently developed and used in each unit structure, resources are wasted, costs are increased, and time becomes inefficient.

In contrast, in an embodiment of the present disclosure, a system is constructed based on the microservice architecture so that results constructed for each stage can be integrated. Accordingly, there are advantages in that whether a candidate for a new material in the molecular structure stage is also suitable in the microstructure stage and whether a material that is suitable in the microstructure stage is also suitable even in the cell (or system) structure stage can be simultaneously determined.

In particular, in the microstructure stage, the adsorption, transmission, and porosity characteristics of a developed material within a microstructure can be accurately checked by using the combination modeling scheme capable of combining computational analysis results, such as computer-aided engineering, and actual experiment results.

Furthermore, an embodiment of the present disclosure provides a service framework capable of cross-evaluation with experiment results by using an image-based AI model which may be integrated and operated within an integrated microservice architecture structure.

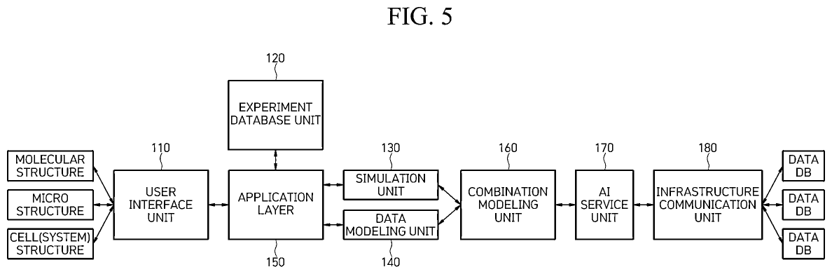

FIG. 5 is a functional block diagram of a virtual material design service framework system 100 according to an embodiment of the present disclosure.

The virtual material design service framework system 100 according to an embodiment of the present disclosure includes a user interface unit (or a user interface) 110, an experiment database unit (or an experiment database) 120, a simulation unit (or a simulation processor) 130, a data modeling unit (or a data modeling processor) 140, an application unit (or an application processor) 150, a combination modeling unit (or a combination modeling processor) 160, an AI service unit (or an AI service processor) 170, and an infrastructure communication unit (or an infrastructure communication interface) 180.

The user interface unit 110 receives information on a new material having at least one structure of a molecular structure, a microstructure, and a cell structure.

The experiment database unit 120 stores experiment data information corresponding to the information on the new material.

The simulation unit 130 generates a simulation model by performing simulations on the received information on the new material. The data modeling unit 140 generates a data model based on the experiment data information.

According to an embodiment of the present disclosure, in the structure of FIG. 5, the user interface unit 110, the experiment database unit 120, the simulation unit 130 and the data modeling unit 140 may be managed through the application unit 150.

When the simulation module and the data model are generated, the combination modeling unit 160 generates a combination model by combining the simulation module and the data model according to the combination modeling scheme.

The AI service unit 170 converts the combination model into an image, and derives a candidate material based on a predetermined image recognition scheme. In this case, the candidate material may be divided into a primary candidate material and a secondary candidate material that is derived from the primary candidate material.

Specifically, the AI service unit 170 may train and apply a predetermined AI model for deriving a candidate material through a predetermined image recognition scheme (e.g., the analysis of an interface, a structure, segmentation, permeability, or an adsorption rate). In this case, the trained AI model may be transmitted to an external cloud server through the infrastructure communication unit 180 that is connected to an AI service.

Furthermore, as an embodiment, the AI service unit 170 may determine whether information on a candidate material corresponding to the molecular structure is suitable when a candidate material corresponding to the microstructure is generated, and may determine whether information on the candidate material corresponding to the microstructure is suitable when a candidate material corresponding to the cell structure is generated.

That is, in a conventional technology, unit structures operate in different systems through the respective application units. In an embodiment of the present disclosure, the simulation model, the data model, the combination model, an image, and a candidate material corresponding to each of the molecular structure, the microstructure, and the cell structure can be checked in parallel. There is an advantage in that determinations of suitability between unit structures can also be performed in parallel.

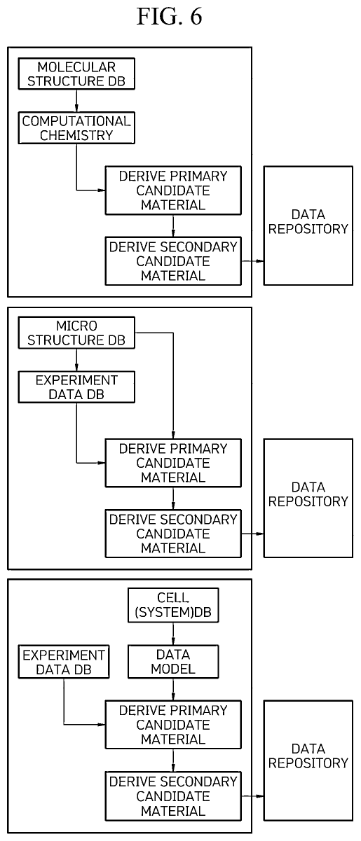

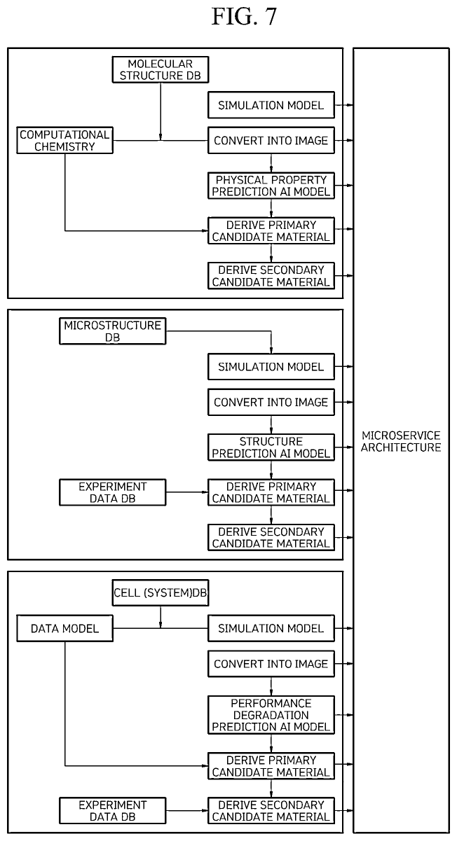

FIGS. 6 and 7 are diagrams for describing an aspect in which data are used in a conventional technology and an embodiment of the present disclosure.

For example, in the case of the molecular structure unit, in a conventional technology, it is impossible to check the characteristics of data from various aspects because the results of the data are simply represented in numbers when computational chemistry is used in discovering a new molecular structure using computational chemistry and the simulation model is not used.

In contrast, an embodiment of the present disclosure has an advantage in that the accuracy of deriving a primary candidate material is increased because various schemes for image recognition (e.g., an interface, a structure, or segmentation) can be used in the AI service unit within the microservice architecture by using meta data on which results are simultaneously checked through the simulation model and the existing computational chemistry. Furthermore, there is an advantage in that the results of the characteristics can be checked in parallel because the results of the candidate material can be immediately transmitted to a next stage.

As another example, in an embodiment of the present disclosure, in the case of the microstructure unit, a three-dimensional microstructure may be obtained by directly applying data stored in the database when the simulation model is generated. There is an advantage in that the accuracy of deriving a primary candidate material is increased because various schemes for image recognition (e.g., an interface, a structure, segmentation, permeability, or an adsorption rate) can be used in the AI service unit within the microservice architecture as in the molecular unit structure. Likewise, there is an advantage in that the results of the characteristics can be checked in parallel because the results of the candidate material can be immediately transmitted to a next stage.

As another example, in an embodiment of the present disclosure, in the case of the cell structure unit, the data model and the simulation model may be used to fill insufficient data or to remove abnormal data of the data model compared to simulation results by combining the data model and the simulation model according to the combination modeling scheme. In this case, the simulation model has an advantage in that it can advance a performance deterioration prediction model by using image data.

In an embodiment of the present disclosure, the AI service unit provided within the microservice architecture may be operated by including a plurality of AI models. That is, the AI service unit may include a first AI model for predicting a physical property based on an image of the molecular structure by the combination model, a second AI model for predicting a structure based on an image of the microstructure by the combination model, and a third AI model for predicting performance deterioration based on an image of the cell structure by the combination model.

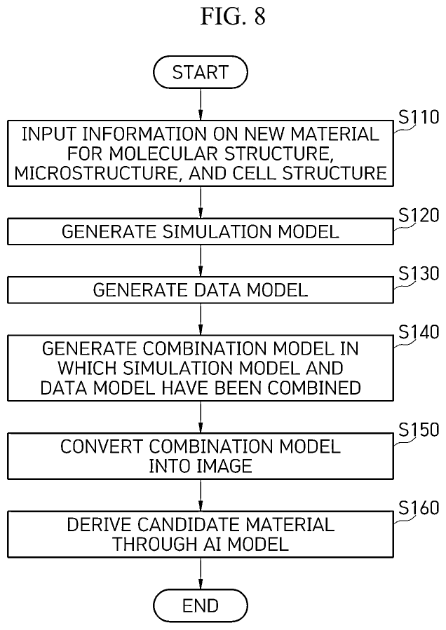

FIG. 8 is a flowchart of an operating method of a virtual material design service framework according to an embodiment of the present disclosure.

First, when information on a new material having at least one structure of a molecular structure, a microstructure, and a cell structure is received (S110), a simulation model is generated by performing simulations on the received information on the new material (S120).

Furthermore, a data model is generated based on experiment data information corresponding to the information on the new material (S130).

Next, a combination model is generated by combining the simulation model and the data model according to the combination modeling scheme (S140). The combination model is converted into an image (S150). A candidate material is derived through a pre-trained AI model to which the predetermined image recognition scheme has been applied (S160).

In the aforementioned description, each of steps S110 to S160 may be further divided into additional steps or the steps may be combined into smaller steps depending on an implementation example of the present disclosure. Furthermore, some of the steps may be omitted, if necessary, and the sequence of the steps may be changed. Furthermore, the contents of FIGS. 1 to 7 and the contents of FIG. 8 are mutually applied.

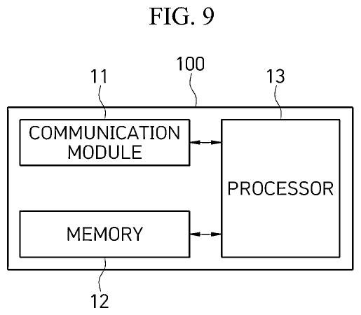

FIG. 9 is a block diagram of a construction of the virtual material design service framework system 100 according to an embodiment of the present disclosure.

The virtual material design service framework system 100 according to an embodiment of the present disclosure includes a communication module 11, memory 12, and a processor 13.

The communication module 11 receives a user input through a user interface, and transmits a trained AI model to an external cloud server. The communication module 11 may include both a wired communication module and a wireless communication module. The wired communication module may be implemented as a power line communication device, a telephone line communication device, cable home (MoCA), Ethernet, IEEE1294, an integrated wired home network, or an RS-485 controller. Furthermore, the wireless communication module may be constructed as a module for implementing a function, such as a wireless LAN (WLAN), Bluetooth, an HDR WPAN, UWB, ZigBee, impulse radio, a 60 GHz WPAN, binary-CDMA, a wireless USB technology, a wireless HDMI technology, 5th generation (5G) communication, long term evolution-advanced (LTE-A), long term evolution (LTE), or wireless fidelity (Wi-Fi).

The memory 12 stores a pre-trained AI model and a database. The processor 13 executes the program stored the memory 12. In this case, the memory 12 commonly refers to a nonvolatile storage device that retains information stored therein although power is not supplied to the nonvolatile storage device and a volatile storage device.

For example, the memory 12 may include NAND flash memory such as a compact flash (CF) card, a secure digital (SD) card, a memory stick, a solid-state drive (SSD), and a micro SD card, a magnetic computer memory device such as a hard disk drive (HDD), and an optical disc drive such as CD-ROM and DVD-ROM.

The processor 13 combines the simulation model and the data model according to the combination modeling scheme, converts the combination model into an image, and then derives a candidate material based on a predetermined image recognition scheme, by executing a program stored in the memory 12.

The operating method of the virtual material design service framework according to an embodiment of the present disclosure may be implemented in the form of a program (or application) in order to be executed by being combined with a computer, that is, hardware, and may be stored in a medium.

The aforementioned program may include a code coded in a computer language, such as C, C++, JAVA, or a machine language which is readable by a processor (CPU) of a computer through a device interface of the computer in order for the computer to read the program and execute the methods implemented as the program. Such a code may include a functional code related to a function, etc. that defines functions necessary to execute the methods, and may include an execution procedure-related control code necessary for the processor of the computer to execute the functions according to a given procedure. Furthermore, such a code may further include a memory reference-related code indicating at which location (address number) of the memory inside or outside the computer additional information or media necessary for the processor of the computer to execute the functions needs to be referred. Furthermore, if the processor of the computer requires communication with any other remote computer or server in order to execute the functions, the code may further include a communication-related code indicating how the processor communicates with the any other remote computer or server by using a communication module of the computer and which information or media needs to be transmitted and received upon communication.

The stored medium means a medium, which semi-permanently stores data and is readable by a device, not a medium storing data for a short moment like a register, cache, or a memory. Specifically, examples of the stored medium include ROM, RAM, CD-ROM, a magnetic tape, a floppy disk, optical data storage, etc., but the present disclosure is not limited thereto. That is, the program may be stored in various recording media in various servers which may be accessed by a computer or various recording media in a computer of a user. Furthermore, the medium may be distributed to computer systems connected over a network, and a code readable by a computer in a distributed way may be stored in the medium.

The description of the present disclosure is illustrative, and a person having ordinary knowledge in the art to which the present disclosure pertains will understand that the present disclosure may be easily modified in other detailed forms without changing the technical spirit or essential characteristic of the present disclosure. Accordingly, it should be construed that the aforementioned embodiments are only illustrative in all aspects, and are not limitative. For example, elements described in the singular form may be carried out in a distributed form. Likewise, elements described in a distributed form may also be carried out in a combined form.

Although the embodiments of the present disclosure have been described with reference to the accompanying drawings, a person of ordinary knowledge in the art to which the present disclosure pertains may understand that the present disclosure may be implemented in other detailed forms without changing the technical spirit or essential characteristics of the present disclosure. Accordingly, it is to be understood that the aforementioned embodiments are only illustrative, but are not limitative in all aspects.

What is claimed is:

1. A virtual material design service framework system for developing a material for a secondary battery based on a microservice architecture, the virtual material design service framework system comprising:
a user interface configured to receive information on a new material having at least one structure of a molecular structure, a microstructure, and a cell structure;
an experiment database configured to store experiment data information corresponding to the information on the new material;
a simulation processor configured to generate a simulation model by performing simulations on the received information on the new material;
a data modeling processor configured to generate a data model based on the experiment data information;
a combination modeling processor configured to combine the simulation model and the data model according to a combination modeling scheme; and
an artificial intelligence service processor configured to convert the combination model into an image and to derive a candidate material based on a predetermined image recognition scheme.

2. The virtual material design service framework system of claim 1, wherein the user interface, the experiment database processor, the simulation processor, and the data modeling processor are configured to be managed through an application processor.

3. The virtual material design service framework system of claim 1, wherein:
the artificial intelligence service processor is configured to train a predetermined artificial intelligence model for deriving the candidate material through the image recognition scheme, and
the artificial intelligence model is configured to be transmitted to an external cloud server through an infrastructure communication interface connected to the artificial intelligence service processor.

4. The virtual material design service framework system of claim 1, wherein the simulation model, the data model, the combination model, the image, and the candidate material corresponding to each of the molecular structure, the microstructure, and the cell structure are configured to be checked in parallel.

5. The virtual material design service framework system of claim 1, wherein the artificial intelligence service processor is configured to:
determine whether information on a candidate material corresponding to the molecular structure is suitable when a candidate material corresponding to the microstructure is generated, and
determine whether information on the candidate material corresponding to the microstructure is suitable when a candidate material corresponding to the cell structure is generated.

6. The virtual material design service framework system of claim 1, wherein the artificial intelligence service processor comprises:
a first artificial intelligence model configured to predict a physical property based on an image of the molecular structure by the combination model,
a second artificial intelligence model configured to predict a structure based on an image of the microstructure by the combination model, and
a third artificial intelligence model configured to predict performance deterioration based on an image of the cell structure by the combination model.

7. An operating method of a virtual material design service framework for developing a material for a secondary battery based on a microservice architecture, the operating method comprising:
receiving information on a new material having at least one structure of a molecular structure, a microstructure, and a cell structure;
generating a simulation model by performing simulations on the received information on the new material;
generating a data model based on experiment data information corresponding to the information on the new material;
generating a combination model by combining the simulation model and the data model according to a combination modeling scheme; and
converting the combination model into an image and deriving a candidate material through a pre-trained artificial intelligence model to which a predetermined image recognition scheme has been applied.

8. The operating method of claim 7, wherein the simulation model, the data model, the combination model, the image, and the candidate material corresponding to each of the molecular structure, the microstructure, and the cell structure are checked in parallel.

9. The operating method of claim 7, wherein deriving the candidate material through the pre-trained artificial intelligence model comprises:
determining whether information on a candidate material corresponding to the molecular structure is suitable when a candidate material corresponding to the microstructure is generated, and
determining whether information on the candidate material corresponding to the microstructure is suitable when a candidate material corresponding to the cell structure is generated.

10. The operating method of claim 7, wherein deriving the candidate material through the pre-trained artificial intelligence model comprises deriving the candidate material based on a first artificial intelligence model for predicting a physical property based on an image of the molecular structure by the combination model, a second artificial intelligence model for predicting a structure based on an image of the microstructure by the combination model, and a third artificial intelligence model for predicting performance deterioration based on an image of the cell structure by the combination model.
